(19) United States
(12) Patent Application Publication
(10) Pub. No.: US 2009/0269287 A1
(43) Pub. Date: Oct. 29, 2009

(54) Oral Care Compositions

(76) Inventor: James Albert Berta, West Chester, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/430,540

(22) Filed: Apr. 27, 2009

Related U.S. Application Data
(60) Provisional application No. 61/048,256, filed on Apr. 28, 2008.

(51) Int. Cl. A61K 8/73; A61K 8/21; A61K 8/24

(52) U.S. Cl. 424/52; 424/49; 424/57

(57) ABSTRACT

Disclosed are oral care compositions, particularly thickened dentifrices in liquid, paste or gel form comprising a binding/thickening system that also function effectively as humectant agent thereby replacing a significant portion or all of traditional humectant components such as glycerin, sorbitol and other polyols. The binding/thickening system comprise select carrageenans that provide a water viscosity of at least about 20 mPa·s in a 1.5% solution at 25° C. and effective water-binding capacity to prevent significant water loss from the composition when exposed to air to cause unacceptable drying out. For example, the water-binding capacity of the carrageenan must be effective such that there is no more than about 0.75% water loss from a dentifrice composition when exposed to air for 30 minutes at room temperature conditions and 50% relative humidity. The present dentifrice compositions exhibit increased dispersibility in saliva during use, which provides for increased contact time of the composition with the user's teeth and oral cavity tissues such that the active dental agents contained therein are more rapidly available to effect their beneficial activity.

### CROSS REFERENCE TO RELATED APPLICATIONS

This application claims the benefit of U.S. Provisional Application No. 61/048,256, filed on Apr. 28, 2008.

### TECHNICAL FIELD

The present invention relates to improved oral care compositions. In particular the invention provides oral care compositions such as dentifrices containing as binder and thickener certain carrageenans, which surprisingly have been found to also have effective humectant properties. The combination of thickening and humectant properties in a single component improves the product and simplifies formulation by making it possible to significantly reduce or even eliminate the content of standard dentifrice humectants, typically polyol compounds such as glycerin, sorbitol, xylitol, maltitol, polyethylene glycol and polypropylene glycol. More significantly, the dentifrice compositions are improved in terms of being easily dispersible during use in an aqueous environment, i.e., saliva, which provides for greater availability and increased uptake of the active ingredients contained therein by the teeth and other oral cavity tissues.

### BACKGROUND OF THE INVENTION

Oral care products such as dentifrice and mouthrinse are routinely used by consumers as part of their oral care hygiene regimens to provide both therapeutic and cosmetic hygiene benefits. Therapeutic benefits include caries prevention which is typically delivered through the use of various fluoride salts; gingivitis prevention by the use of an antimicrobial agent such as triclosan, stannous fluoride, or essential oils; or hypersensitivity control through the use of ingredients such as stannous fluoride, strontium chloride or potassium nitrate. Hygiene and cosmetic benefits provided by oral care products include the control of plaque and calculus formation, removal and prevention of tooth stain, tooth whitening, breath freshening, and overall improvements in mouth feel impression which can be broadly characterized as mouth feel aesthetics. Thus oral care products for daily use such as dentifrice and rinses require multiple actives and additives working by different mechanisms to provide the complete range of therapeutic and aesthetic benefits.

Toothpastes, the most popular form of dentifrice, typically contain an abrasive, a humectant system, a surfactant, thickening or binding agents such as gums or resins to provide cohesiveness and structure, flavor, color, a solvent such as water and alcohol and other agents for specific effects. Other than water, the predominant components of toothpastes are the abrasive and the humectant system. For example, in recent years the most common dentifrices in the market contain 10% or higher abrasive and 20% or higher level up to about 80% of humectants.

The humectant serves to keep toothpaste compositions from hardening or crystallizing upon exposure to air, to give compositions a moist feel to the mouth, and, for particular humectants, to impart desirable sweetness. The humectants comprise one or more liquids which along with water and/or other solvents make up the carrier phase in which other toothpaste ingredients particularly insoluble abrasives are dispersed to provide a stable paste. An important function of humectants is to slow the toothpaste from drying out due to evaporation of water or other volatile materials when the toothpaste package is left open or during use of product by the consumer. A problem that can arise with toothpaste products packaged in squeeze tubes is the cementing of the cap to the tube during use of the product by the consumer. This can occur, for example, when toothpaste unintentionally gets onto the threads of the tube and the tube is left undisturbed for a long period of time. This problem is called cap lock and is due to the crystallization of solid material in the toothpaste. In order to prevent cap lock, humectants are added to toothpastes in order to preserve moisture and prevent crystallization. A related problem is caused by the cap not being replaced between uses. The paste in the nozzle of the tube can dry out if appropriate humectants are not present in the toothpaste in effective amounts. Dental rinses are also formulated with humectants because of the mouth feel and taste benefits they provide. Typical humectants that have been used in dentifrice and rinse compositions include edible polyhydric alcohols such as glycerin, sorbitol, xylitol, propylene glycol, butylene glycol, polyethylene glycol and polypropylene glycol.

The most commonly used liquid humectants are glycerin, 70% aqueous sorbitol and mixtures thereof. One advantage of glycerin and sorbitol is that they are essentially not fermentable by cariogenic bacteria. Other humectants that have been suggested such as maltodextrins are fermentable by cariogenic bacteria and are thus less suitable. Another advantage of glycerin and sorbitol is the ability to be formulated in clear or translucent gels by virtue of having a refractive index similar to the refractive index of the abrasive component, particularly silica. In both opaque pastes and transparent gels, the level of glycerin and/or sorbitol must remain relatively high to effectively inhibit drying out of the composition and provide the mouth feel benefits. High levels of humectant, particularly glycerin and sorbitol are undesirable because of high and rapidly increasing cost. Significant cost reduction can be achieved if part or all of the humectant(s) can be eliminated.

The present invention is based on the discovery that certain binders or thickening agents have humectant properties and can thus replace at least part of the polyols particularly glycerin and sorbitol in oral care compositions. The main purpose of binders is to thicken toothpastes to a desirable consistency and to prevent separation of the solid and liquid components, especially during storage. They also affect the speed and volume of foam production, the rate of flavor release and product dispersibility, the appearance of the toothpaste ribbon on the toothbrush, and the rinseability from the toothbrush. Commonly used binders include one or a combination of carrageenan, carboxyvinyl polymers, hydroxyethyl cellulose (HEC), natural and synthetic clays (e.g., Veegum and Laponite) and water soluble salts of cellulose ethers such as sodium carboxymethylcellulose (CMC) and sodium carboxymethyl hydroxyethyl cellulose. Natural gums such as gum karaya, xanthan gum, gum arabic, and gum tragacanth can also be used. Colloidal magnesium aluminum silicate or finely divided silica has also been used as part of the binder system to further improve texture.

In one aspect, the present invention provides oral care products comprising certain carrageenans which function effectively as binder and humectant thus replacing a significant portion or all of the polyol humectant component, simplifying formulation and reducing cost, while maintaining desired properties attributed to the humectant including moisture retention, and texture, mouthfeel and taste benefits. The present compositions are improved in terms of enhanced dispersibility or solubility in saliva during use.

### SUMMARY OF THE INVENTION

The present invention is directed to oral care compositions, particularly thickened dentifrices in liquid, paste or gel form comprising a binding/thickening system that also function effectively as humectant agent thereby replacing a significant portion or all of traditional humectant components such as glycerin, sorbitol and other polyols. The binding/thickening system comprise select carrageenans that provide a water viscosity of at least about 20 mPa·s in a 1.5% solution at 25° C. and effective water-binding capacity to prevent significant water loss from the composition when exposed to air to cause unacceptable drying out. For example, the water-binding capacity of the carrageenan must be effective such that there is no more than about 0.75% water loss from the dentifrice composition when exposed to air for 30 minutes at room temperature conditions and 50% relative humidity. The dentifrice compositions exhibit increased dispersibility in saliva during use, which provides for increased contact time of the composition with the user's teeth and oral cavity tissues such that the active dental agents contained therein are more rapidly available to effect their beneficial activity.

These and other features, aspects, and advantages of the present invention will become evident to those skilled in the art from the detailed description which follows.

### DETAILED DESCRIPTION OF THE INVENTION

While the specification concludes with claims particularly pointing out and distinctly claiming the invention, it is believed that the present invention will be better understood from the following description.

All percentages and ratios used hereinafter are by weight of total composition, unless otherwise indicated. All percentages, ratios, and levels of ingredients referred to herein are based on the actual amount of the ingredient, and do not include solvents, fillers, or other materials with which the ingredient may be combined as a commercially available product, unless otherwise indicated.

All measurements referred to herein are made at room temperature of about 25° C. unless otherwise specified.

Herein, “comprising” means that other steps and other components which do not affect the end result can be added. This term encompasses the terms “consisting of” and “consisting essentially of.”

As used herein, the word “include,” and its variants, are intended to be non-limiting, such that recitation of items in a list is not to the exclusion of other like items that may also be useful in the materials, compositions, devices, and methods of this invention.

As used herein, the words “preferred”, “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

By “oral care composition” is meant a product, which in the ordinary course of usage, is not intentionally swallowed for purposes of systemic administration of particular therapeutic agents, but is rather retained in the oral cavity for a time sufficient to contact substantially all of the dental surfaces and/or oral tissues for purposes of oral activity. The oral care composition may be in various forms including toothpaste, dentifrice, tooth gel, subgingival gel, mouthrinse, mousse, foam, denture product, mouthspray, lozenge, chewable tablet or chewing gum. The oral care composition may also be incorporated onto strips or films for direct application or attachment to oral surfaces.

The term “dentifrice”, as used herein, includes paste, gel, liquid, powder or tablet formulations unless otherwise specified. The dentifrice composition may be a single phase composition or may be a combination of two or more separate dentifrice compositions. The dentifrice composition may be in any desired form, such as deep striped, surface striped, multilayered, having a gel surrounding a paste, or any combination thereof. Each dentifrice composition in a dentifrice comprising two or more separate dentifrice compositions may be contained in a physically separated compartment of a dispenser and dispensed side-by-side.

The term “dispenser”, as used herein, means any pump, tube, or container suitable for dispensing compositions such as dentifrices.

The term “teeth”, as used herein, refers to natural teeth as well as artificial teeth or dental prosthesis.

The term “orally-acceptable carrier” refer to safe and effective materials and conventional additives used in oral care compositions including but not limited to one or more of fluoride ion sources, anti-calculus or anti-tartar agents, antimicrobial agents, buffers, abrasives such as silica, alkali metal bicarbonate salts, thickening materials, humectants, water, surfactants, titanium dioxide, flavor system, sweetening agents, xylitol, and coloring agents.

Active and other ingredients useful herein may be categorized or described by their cosmetic and/or therapeutic benefit or their postulated mode of action or function. However, it is to be understood that the active and other ingredients useful herein can, in some instances, provide more than one cosmetic and/or therapeutic benefit or function or operate via more than one mode of action. Therefore, classifications herein are made for the sake of convenience and are not intended to limit an ingredient to the particularly stated application or applications listed.

Herein, the terms “tartar” and “calculus” are used interchangeably and refer to mineralized dental plaque biofilms.

The essential and optional components of the present compositions are described in the following paragraphs.

### Thickening/Binding System

The present invention is based on selected carrageenans that provide thickening/binding as well as humectant functions in oral care compositions, particularly dentifrices. The use of carrageenans in dentifrice formulations is not new as carrageenans particularly of molecular weights well in excess of 100,000, and generally in the molecular weight range of 250,000 to 500,000, have been used to impart viscosity and rheological characteristics important for dentifrices including desirable consistency, texture, stand-up, physical stability and low stringiness. Carrageenans of molecular weights below 100,000 have also been reported and are suitable for use typically at higher levels because of their lower viscosity building capability. However, the use of carrageenans as replacement for humectants in dentifrice formulations has not been appreciated.

Carrageenans belong to a family of hydrocolloid polysaccharides derived from seaweed. There are at least five carrageenan polymers, and three—lambda, kappa and iota—are typically used in dentifrice applications. All carrageenans contain repeating galactose units joined by alternating β1→3 and α1→4 glycosidic linkages and are partially sulfated. The types of carrageenans may be distinguished, in part, by their degree of sulfation. Kappa carrageenan has a repeating unit of D-galactose-4-sulfate-3,6-anhydro-D-galactose providing a sulfate ester content of about 18 to 25%. Iota carrageenan has a repeating unit of D-galactose-4-sulfate-3,6-anhydro-D-galactose-2-sulfate providing a sulfate ester content of about 25 to 34%. Lambda carrageenan has a repeating unit of D-galactose-2-sulfate-D-galactose-2,6-disulfate providing a sulfate ester content of about 30 to 40%. Carrageenans can increase the viscosity or form a network of molecules to provide a gel texture. Carrageenans generally form gels that are thixotropic. Such gels are reported to exhibit excellent extrudability, flavor release and rinseability. The use of kappa and iota carrageenan as binders in gel toothpaste is known particularly to provide a toothpaste that is non-stringy and has a low syneresis problem; i.e., prevent separation of water from the gel. Toothpaste products are deemed unacceptable if they are not stable against such phase separation. Commercial supplies of carrageenan for particular applications may be characterized in terms of the ratio of iota, kappa and lambda forms. For toothpaste applications, the kappa and iota forms of carrageenan are used to obtain fairly firm gels that remain firm at room temperature yet soften at or near body temperature to facilitate dispersion of toothpaste components such as actives, flavor and coolants in the mouth during use.

The formulation properties of toothpastes will depend on the properties of the binder, abrasive, humectant, water and other components of the formulation, and they will also depend on how these components behave in complex mixtures with each other. Thus there is a tremendous advantage in being able to formulate without or with significantly lower levels of humectant in that potential interactions are diminished. Further, the reduction in cost is significant since humectants are typically used at fairly high levels.

Suitable carrageenans functioning as binder and humectant replacement can be characterized in terms of their water binding capacity, i.e., the ability to absorb and retain water, thereby reducing the amount of free water available in the product. Suitable carrageenans will have water binding capacity at their typical use level of about 0.1% to 10% comparable to that provided by humectants at their use level. For example, glycerin, sorbitol and mixtures are typical humectants used in dentifrice formulations at levels ranging from 10% to 30%. Glycerin is reported to have a water binding capacity of better than 3× its weight. Thus, at the typical amounts of glycerin used in dentifrices, it is estimated that most if not all of the water content of the dentifrice will be bound. If more of the water content is bound or not free, the less susceptible is the product to drying out when exposed to air or conditions wherein free water can quickly evaporate. Suitable carrageenans are supplied by FMC Biopolymer under the tradename Viscarin®, such as one having a 1.5% water viscosity greater than about 20 mPa·s, 1.5% solution pH ranging from 7.0 to 11.0, moisture content <15% and particle size such that >90% pass through USSS 150 μm. Examples of suitable grades include Viscarin® TP389, Viscarin® RE-IN-0907-02A, Viscarin® RE-IN-0308-01 and Viscarin® 1280. CP Kelco is another supplier of carrageenan under the Genuvisco® tradename.

The susceptibility to drying out of the present compositions using carrageenans as water binding agent is comparable to compositions comprising humectants. This property was evaluated by measuring weight loss due to evaporation using the following method.
(1) Weigh 25 grams of test paste in a 30 ml sealable cup or container.
(2) Weigh the cup+paste uncovered.
(3) Place the uncovered cup in a 25° C. 50% RH (relative humidity) controlled environment for 30 minutes.
(4) Weigh the cup+paste after the 30 minute evaporation.
(5) Calculate the % weight lost.

The present composition without humectant had comparable performance with regard to water loss (Example 1A below; 0.68% water loss) to a high humectant-containing product (Blend-A-Med Cavity Protection with 30% sorbitol added as 70% solution, 0.63% water loss). By comparison, a system with no humectant and no carrageenan had 1.0% water loss over the same time period. Such water loss for the present carrageenan formulation is minimal, demonstrating that its water binding capacity is equivalent to that provided by a humectant, thereby slowing unacceptable drying up of the product.

Thickening agents other than the subject carrageenans may be used in the present compositions. Suitable thickening agents include one or a combination of carboxyvinyl polymers, hydroxyethyl cellulose (HEC), natural and synthetic clays (e.g., Veegum and Laponite) and water soluble salts of cellulose ethers such as sodium carboxymethylcellulose (CMC) and sodium carboxymethyl hydroxyethyl cellulose. Natural gums such as gum karaya, xanthan gum, gum arabic, and gum tragacanth can also be used. Colloidal magnesium aluminum silicate or finely divided silica can be used as part of the thickening agent to further improve texture and or achieve a desired viscosity.

Suitable carboxyvinyl polymers useful as thickening or gelling agents include carbomers which are homopolymers of acrylic acid crosslinked with an alkyl ether of pentaerythritol or an alkyl ether of sucrose. Carbomers are commercially available from B. F. Goodrich as the Carbopol® series, including Carbopol 934, 940, 941, 956, and mixtures thereof.

The total amount of thickening agents will typically range from about 0.1% to about 15%, preferably from about 1% to about 10%, more preferably from about 2% to about 8%, by weight of the total toothpaste or gel composition.

The present dentifrice compositions will typically have a viscosity of 500 Pa·s or more, preferably from 500-4000 Pa·s measured at 1/s and 25° C.

Optionally some humectant may be included to improve sensory properties of the compositions including texture, mouthfeel and sweetness. The added humectant, on a pure humectant basis, will generally be 10% or less, preferably about 5% or less by weight. Suitable humectants for use in compositions of the subject invention include edible polyhydric alcohols such as glycerin, sorbitol, xylitol, butylene glycol, polyethylene glycol and propylene glycol. Trimethyl glycine may also be used.

Importantly, the present compositions formulated with little or no humectant exhibit increased dispersibility or solubility in aqueous medium, such as saliva. By dispersibility is meant the ability of the toothpaste or dentifrice to mix and disperse with saliva. Increased dispersibility means the toothpaste or dentifrice can quickly spread across the teeth and into crevices to provide faster cleaning and treatment of the teeth and surrounding tissues. Because most people on average brush for 60 seconds or less, it is advantageous that the active ingredients are delivered within that time frame to the person's teeth and other oral cavity surfaces. Otherwise, their intended benefit may not be realized to the extent desired. In addition, increased dispersibility of the present formulations ensures that the entire amount of product dispensed for use is utilized during the brushing process, i.e., little or none precipitate out and are expectorated after brushing. In consumer tests, the present formulations are judged to have a light creamy texture and quick dispersibility characteristics. Consumers described dispersibility in terms of attributes including ease of product spreading in the mouth during brushing, ease of rinsing and in the amount of unused undispersed product. The texture and quick dispersion characteristics of the present dentifrice drove consumer preference, being associated with deeper and more thorough cleaning, less waste since essentially no residue of unused paste was expectorated or left in the mouth or brush and suitability for the whole family especially small children who could have a more complete cleaning in less time without the struggle to dissolve the toothpaste.

Dispersibility characteristics of the present compositions compared with conventional products was determined using the following method which measures the time it takes to disperse a sample such as a dentifrice in a low-shear, aqueous environment. The longer the time, the less dispersible is the product. The dispersion times measured using the present method correlate with the dispersibility characteristics exhibited by the dentifrice under brushing conditions in the mouth. For example, a dentifrice containing 1.5% carrageenan and no humectant (Example 2A below) was described in consumer testing as quickly dispersing during brushing.

A USP dissolution bath vessel (Van Kel V7000) equipped with paddle attachments is used to disperse a quantity of product in 37° C. distilled water simulating an aqueous environment at body temperature. All aspects that contribute to dispersion (temperature, water volume, mixing speed, product sample size) are controlled to allow for the generation of reproducible results. Visual detection is used to judge the endpoint of sample dispersion. The method is conducted as follows:
(1) Fill vessels with 900 mL of distilled water. Set temperature control for 37° C. and allow vessels to equilibrate with bath temperature. Verify the vessel temperature with a calibrate thermometer. This should take approximately 15-20 minutes.
(2) Using a marked transfer tube, aspirate and dispense 2.5 cm of sample into the bottom of the vessel, making sure that the samples do not contain large amounts of air. This can be avoided by overfilling the transfer tube and dispensing to the line. Dispense the sample as one large bolus; this is most easily achieved by dispensing the sample quickly from the transfer tube.
(3) Once all of samples are dispensed, initiate mixing at 50 rpm and start the stopwatch. Observe products for dispersion and record time when no apparent changes occur for a particular sample, i.e., entire sample is dispersed or any remaining sample no longer disperses.

In one embodiment, the compositions of the subject invention are dentifrices in paste, gel or liquid forms. Components of such products generally include the present carrageenans as thickening agent and humectant (from 0% up to about 10%), one or more of a dental abrasive (from about 6% to about 50%), a surfactant (from about 0.5% to about 10%), a flavoring agent (from about 0.04% to about 2%), a sweetening agent (from about 0.1% to about 3%), a coloring agent (from about 0.01% to about 0.5%) and water (up to 90%). Dentifrices may also include one or more of an anticaries agent (such as from about 0.05% to about 0.3% as fluoride ion) and an anticalculus agent (from about 0.1% to about 13%).

Other embodiments of the subject invention are liquid products, including mouthwashes or rinses, mouth sprays, dental solutions and irrigation fluids wherein the humectant component is replaced by carrageenan. Components of such mouthwashes and mouth sprays typically include one or more of water (from about 45% to about 95%), ethanol (from about 0% to about 25%), a surfactant (from about 0.01% to about 7%), a flavoring agent (from about 0.04% to about 2%), a sweetening agent (from about 0.1% to about 3%), and a coloring agent (from about 0.001% to about 0.5%). Such mouthwashes and mouth sprays may also include one or more of an anticaries agent (from about 0.05% to about 0.3% as fluoride ion) and an anticalculus agent (from about 0.1% to about 3%). Components of dental solutions generally include one or more of water (from about 90% to about 99%), preservative (from about 0.01% to about 0.5%), flavoring agent (from about 0.04% to about 2%), sweetening agent (from about 0.1% to about 3%), and surfactant (from 0% to about 5%).

The components of the subject formulations are collectively referred to as orally acceptable carriers or excipients and are discussed in the following paragraphs.

Orally acceptable carrier materials include one or more compatible solid or liquid excipients or diluents which are suitable for topical oral administration. By “compatible” is meant that the components of the composition are capable of being commingled without interaction in a manner which would substantially reduce composition stability and/or efficacy.

The carriers or excipients suitable for the preparation of compositions of the present invention are well known in the art. Their selection will depend on secondary considerations like taste, cost, and shelf stability, etc.

### Abrasives

Dental abrasives useful in the compositions of the subject invention include many different materials. The material selected must be one which is compatible within the composition of interest and does not excessively abrade dentin. Suitable abrasives include, for example, silicas including gels and precipitates, insoluble sodium polymetaphosphate, hydrated alumina, calcium carbonate, dicalcium orthophosphate dihydrate, calcium pyrophosphate, tricalcium phosphate, calcium polymetaphosphate, and resinous abrasive materials such as particulate condensation products of urea and formaldehyde.

Another class of abrasives for use in the present compositions is the particulate thermo-setting polymerized resins as described in U.S. Pat. No. 3,070,510 issued to Cooley & Grabenstetter. Suitable resins include, for example, melamines, phenolics, ureas, melamine-ureas, melamine-formaldehydes, urea-formaldehyde, melamine-urea-formaldehydes, cross-linked epoxides, and cross-linked polyesters.

Silica dental abrasives of various types are preferred because of their unique benefits of exceptional dental cleaning and polishing performance without unduly abrading tooth enamel or dentine. The silica abrasive polishing materials herein, as well as other abrasives, generally have an average particle size ranging between about 0.1 to about 30 microns, and preferably from about 5 to about 15 microns. The abrasive can be precipitated silica or silica gels such as the silica xerogels described in Pader et al., U.S. Pat. No. 3,538,230 and DiGiulio, U.S. Pat. No. 3,862,307. Examples include the silica xerogels marketed under the trade name “Syloid” by the W.R. Grace & Company, Davison Chemical Division and precipitated silica materials such as those marketed by the J. M. Huber Corporation under the trade name, Zeodent®, particularly the silicas carrying the designation Zeodent® 119, Zeodent® 118, Zeodent® 109 and Zeodent® 129. The types of silica dental abrasives useful in the toothpastes of the present invention are described in more detail in Wason, U.S. Pat. No. 4,340,583; and in commonly-assigned U.S. Pat. Nos. 5,603,920; 5,589,160; 5,658,553; 5,651,958; and 6,740,311.

Mixtures of abrasives can be used such as mixtures of the various grades of Zeodent® silica abrasives listed above. The total amount of abrasive in dentifrice compositions of the subject invention typically range from about 6% to about 70% by weight; toothpastes preferably contain from about 10% to about 50% of abrasives. Dental solution, mouth spray, mouthwash and non-abrasive gel compositions of the subject invention typically contain little or no abrasive.

### Fluoride Source

The oral compositions of the present invention will optionally include a soluble fluoride source capable of providing bioavailable and efficacious fluoride ions. Soluble fluoride ion sources include sodium fluoride, stannous fluoride, indium fluoride, amine fluoride and sodium monofluorophosphate. Sodium fluoride and stannous fluoride are preferred soluble fluoride sources. Norris et al., U.S. Pat. No. 2,946,725 and Widder et al., U.S. Pat. No. 3,678,154 disclose such fluoride sources as well as others.

The present compositions may contain a soluble fluoride ion source in an amount sufficient to give a fluoride ion concentration in the composition from about 50 ppm to about 3500 ppm, and preferably from about 500 ppm to about 3000 ppm. To deliver the desired amount of fluoride ions, fluoride ion sources may be present in the total oral composition at an amount of from about 0.1% to about 5%, preferably from about 0.2% to about 1%, and more preferably from about 0.3% to about 0.6%, by weight of the total composition delivered to the oral cavity.

### Flavor System

The flavor system is typically added to oral care compositions, to provide a pleasant tasting composition and to effectively mask any unpleasant taste and sensations due to certain components of the composition such as antimicrobial actives or peroxide. Pleasant tasting compositions improve user compliance to prescribed or recommended use of oral care products. The present flavor system will comprise flavor components, in particular those that have been found to be relatively stable in the presence of usual oral care product carrier materials or excipients. The combination of the selected flavoring components with sensate ingredients such as coolant(s) provides a high-impact refreshing sensation with a well-rounded flavor profile.

The flavor system may comprise flavor ingredients including but not limited to peppermint oil, corn mint oil, spearmint oil, oil of wintergreen, clove bud oil, cassia, sage, parsley oil, marjoram, lemon, lime, orange, cis-jasmone, 2,5-dimethyl-4-hydroxy-3(2H)-furanone, 5-ethyl-3-hydroxy-4-methyl-2(5H)-furanone, vanillin, ethyl vanillin, anisaldehyde, 3,4-methylenedioxybenzaldehyde, 3,4-dimethoxybenzaldehyde, 4-hydroxybenzaldehyde, 2-methoxybenzaldehyde, benzaldehyde; cinnamaldehyde, hexyl cinnamaldehyde, alpha-methyl cinnamaldehyde, ortho-methoxy cinnamaldehyde, alpha-amyl cinnamaldehydepropenyl guaethol, heliotropine, 4-cis-heptenal, diacetyl, methyl-ρ-tert-butyl phenyl acetate, menthol, methyl salicylate, ethyl salicylate, 1-menthyl acetate, oxanone, alpha-irisone, methyl cinnamate, ethyl cinnamate, butyl cinnamate, ethyl butyrate, ethyl acetate, methyl anthranilate, iso-amyl acetate, iso-amyl butyrate, allyl caproate, eugenol, eucalyptol, thymol, cinnamic alcohol, octanol, octanal, decanol, decanal, phenylethyl alcohol, benzyl alcohol, alpha-terpineol, linalool, limonene, citral, maltol, ethyl maltol, anethole, dihydroanethole, carvone, menthone, β-damascenone, ionone, gamma decalactone, gamma nonalactone, gamma undecalactone and mixtures thereof. Generally suitable flavoring ingredients are those containing structural features and functional groups that are less prone to redox reactions. These include derivatives of flavor chemicals that are saturated or contain stable aromatic rings or ester groups. Also suitable are flavor chemicals that may undergo some oxidation or degradation without resulting in a significant change in the flavor character or profile. The flavor ingredients may be supplied in the composition as single or purified chemicals or by addition of natural oils or extracts that have preferably undergone a refining treatment to remove components that are relatively unstable and may degrade and alter the desired flavor profile, resulting in a less acceptable product from an organoleptic standpoint. Flavoring agents are generally used in the compositions at levels of from about 0.001% to about 5%, by weight of the composition.

The flavor system will typically include a sweetening agent. Suitable sweeteners include those well known in the art, including both natural and artificial sweeteners. Some suitable water-soluble sweeteners include monosaccharides, disaccharides and polysaccharides such as xylose, ribose, glucose (dextrose), mannose, galactose, fructose (levulose), sucrose (sugar), maltose, invert sugar (a mixture of fructose and glucose derived from sucrose), partially hydrolyzed starch, corn syrup solids, dihydrochalcones, monellin, steviosides, and glycyrrhizin. Suitable water-soluble artificial sweeteners include soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin, and the like. Other suitable sweeteners include dipeptide based sweeteners, such as L-aspartic acid derived sweeteners, such as L-aspartyl-L-phenylalanine methyl ester (aspartame) and materials described in U.S. Pat. No. 3,492,131, L-alpha-aspartyl-N-(2,2,4,4-tetramethyl-3-thietanyl)-D-alaninamide hydrate, methyl esters of L-aspartyl-L-phenylglycerin and L-aspartyl-L-2,5,dihydrophenyl-glycine, L-aspartyl-2,5-dihydro-L-phenylalanine, L-aspartyl-L-(1-cyclohexylen)-alanine, and the like. Water-soluble sweeteners derived from naturally occurring water-soluble sweeteners, such as a chlorinated derivative of ordinary sugar (sucrose), known, for example, under the product description of sucralose as well as protein based sweeteners such as thaumatoccous danielli (Thaumatin I and II) can be used. A composition preferably contains from about 0.1% to about 10% of sweetener, preferably from about 0.1% to about 1%, by weight of the composition.

Suitable cooling agents or coolants include a wide variety of materials such as menthol and derivatives thereof. Among synthetic coolants, many are derivatives of or are structurally related to menthol, i.e., containing the cyclohexane moiety, and derivatized with functional groups including carboxamide, ketal, ester, ether and alcohol. Examples include the ρ-menthanecarboxamide compounds such as N-ethyl-p-menthan-3-carboxamide, known commercially as “WS-3”, and others in the series such as WS-5, WS-11, WS-14 and WS-30. An example of a synthetic carboxamide coolant that is structurally unrelated to menthol is N,2,3-trimethyl-2-isopropylbutanamide, known as “WS-23”. Additional suitable coolants include 3-1-menthoxypropane-1,2-diol known as TK-10, isopulegol (under the tradename Coolact P) and ρ-menthane-3,8-diol (under the tradename Coolact 38D) all available from Takasago; menthone glycerol acetal known as MGA; menthyl esthers such as menthyl acetate, menthyl acetoacetate, menthyl lactate known as Frescolat® supplied by Haarmann and Reimer, and monomenthyl succinate under the tradename Physcool from V. Mane. The terms menthol and menthyl as used herein include dextro- and levorotatory isomers of these compounds and racemic mixtures thereof. TK-10 is described in U.S. Pat. No. 4,459,425, Amano et al. WS-3 and other carboxamide cooling agents are described for example in U.S. Pat. Nos. 4,136,163; 4,150,052; 4,153,679; 4,157,384; 4,178,459 and 4,230,688. Additional N-substituted ρ-menthane carboxamides are described in WO 2005/049553A1 including N-(4-cyanomethylphenyl)-ρ-menthanecarboxamide, N-(4-sulfamoylphenyl)-ρ-menthanecarboxamide, N-(4-cyanophenyl)-ρ-menthanecarboxamide, N-(4-acetylphenyl)-ρ-menthanecarboxamide, N-(4-hydroxymethylphenyl)-ρ-menthanecarboxamide and N-(3-hydroxy-4-methoxyphenyl)-ρ-menthanecarboxamide.

In addition the flavor system may include salivating agents, warming agents, and numbing agents. These agents are present in the compositions at a level of from about 0.001% to about 10%, preferably from about 0.1% to about 1%, by weight of the composition. Suitable salivating agents include Jambu® manufactured by Takasago. Suitable numbing agents include benzocaine, lidocaine, clove bud oil, and ethanol. Examples of warming agents include ethanol, capsicum and nicotinate esters, such as benzyl nicotinate. Use of agents with warming effects may of course alter the cooling effect of coolants and will need to be considered, particularly in optimizing the level of coolants.

### Active Agents

The present compositions may optionally include active agents, such as antimicrobial/anti-plaque agents. Included among such agents are water insoluble non-cationic antimicrobial agents such as halogenated diphenyl ethers, phenolic compounds including phenol and its homologs, mono and poly-alkyl and aromatic halophenols, resorcinol and its derivatives, bisphenolic compounds and halogenated salicylanilides, benzoic esters, and halogenated carbanilides. The water soluble antimicrobials include quaternary ammonium salts and bis-biquanide salts, and triclosan monophosphate. The quaternary ammonium agents include those in which one or two of the substituents on the quaternary nitrogen has a carbon chain length (typically alkyl group) from about 8 to about 20, typically from about 10 to about 18 carbon atoms while the remaining substitutents (typically alkyl or benzyl group) have a lower number of carbon atoms, such as from about 1 to about 7 carbon atoms, typically methyl or ethyl groups. Dodecyl trimethyl ammonium bromide, tetradecylpyridinium chloride, domiphen bromide, N-tetradecyl-4-ethyl pyridinium chloride, dodecyl dimethyl (2-phenoxyethyl) ammonium bromide, benzyl dimethylstearyl ammonium chloride, cetyl pyridinium chloride, quaternized 5-amino-1,3-bis(2-ethyl-hexyl)-5-methyl hexa hydropyrimidine, benzalkonium chloride, benzethonium chloride and methyl benzethonium chloride are exemplary of typical quaternary ammonium antibacterial agents. Other compounds are bis[4-(R-amino)-1-pyridinium] alkanes as disclosed in U.S. Pat. No. 4,206,215, issued to Bailey. Other antimicrobials such as copper salts, zinc salts and stannous salts may also be included. Also useful are enzymes, including endoglycosidase, papain, dextranase, mutanase, and mixtures thereof. Such agents are disclosed in U.S. Pat. No. 2,946,725 to Norris et al. and in U.S. Pat. No. 4,051,234 to Gieske et al. Preferred antimicrobial agents include zinc salts, stannous salts, cetyl pyridinium chloride, chlorhexidine, triclosan, triclosan monophosphate, and flavor oils. Triclosan and other agents of this type are disclosed in Parran, Jr. et al., U.S. Pat. No. 5,015,466 and U.S. Pat. No. 4,894,220 to Nabi et al. These agents provide anti-plaque benefits and are typically present at levels of from about 0.01% to about 5.0%, by weight of the composition.

Still another active agent that may be included in the present compositions is a tooth bleaching active selected from the group consisting of peroxides, perborates, percarbonates, peroxyacids, persulfates, and combinations thereof. Suitable peroxide compounds include hydrogen peroxide, urea peroxide, calcium peroxide, sodium peroxide, zinc peroxide and mixtures thereof. A preferred percarbonate is sodium percarbonate. Preferred persulfates are oxones.

Preferred peroxide sources for use in dentifrice formulations are calcium peroxide and urea peroxide. Hydrogen peroxide and urea peroxide are preferred for use in mouthrinse formulations. The following amounts represent the amount of peroxide raw material, although the peroxide source may contain ingredients other than the peroxide raw material. The present composition may contain from about 0.01% to about 30%, preferably from about 0.1% to about 10%, and more preferably from about 0.5% to about 5% of a peroxide source, by weight of the composition.

Another optional active agent that may be added to the present compositions is a dentinal desensitizing agent to control hypersensitivity, such as salts of potassium, calcium, strontium and tin including nitrate, chloride, fluoride, phosphates, pyrophosphate, polyphosphate, citrate, oxalate and sulfate.

### Anticalculus Agent

The present compositions may optionally include an anticalculus agent, such as a pyrophosphate salt as a source of pyrophosphate ion. The pyrophosphate salts useful in the present compositions include the dialkali metal pyrophosphate salts, tetraalkali metal pyrophosphate salts, and mixtures thereof. Disodium dihydrogen pyrophosphate (Na₂H₂P₂O₇), tetrasodium pyrophosphate (Na₄P₂O₇), and tetrapotassium pyrophosphate (K₄P₂O₇) in their unhydrated as well as hydrated forms are the preferred species. In compositions of the present invention, the pyrophosphate salt may be present in one of three ways: predominately dissolved, predominately undissolved, or a mixture of dissolved and undissolved pyrophosphate.

Compositions comprising predominately dissolved pyrophosphate refer to compositions where at least one pyrophosphate ion source is in an amount sufficient to provide at least about 1.0% free pyrophosphate ions. The amount of free pyrophosphate ions may be from about 1% to about 15%, from about 1.5% to about 10% in one embodiment, and from about 2% to about 6% in another embodiment. Free pyrophosphate ions may be present in a variety of protonated states depending on the pH of the composition.

Compositions comprising predominately undissolved pyrophosphate refer to compositions containing no more than about 20% of the total pyrophosphate salt dissolved in the composition, preferably less than about 10% of the total pyrophosphate dissolved in the composition. Tetrasodium pyrophosphate salt is a preferred pyrophosphate salt in these compositions. Tetrasodium pyrophosphate may be the anhydrous salt form or the decahydrate form, or any other species stable in solid form in the dentifrice compositions. The salt is in its solid particle form, which may be its crystalline and/or amorphous state, with the particle size of the salt preferably being small enough to be aesthetically acceptable and readily soluble during use. The amount of pyrophosphate salt useful in making these compositions is any tartar control effective amount, generally from about 1.5% to about 15%, preferably from about 2% to about 10%, and most preferably from about 3% to about 8%, by weight of the dentifrice composition.

Compositions may also comprise a mixture of dissolved and undissolved pyrophosphate salts. Any of the above mentioned pyrophosphate salts may be used.

The pyrophosphate salts are described in more detail in Kirk-Othmer Encyclopedia of Chemical Technology, Third Edition, Volume 17, Wiley-Interscience Publishers (1982).

Optional agents to be used in place of or in combination with the pyrophosphate salt include such known materials as synthetic anionic polymers, including polyacrylates and copolymers of maleic anhydride or acid and methyl vinyl ether (e.g., Gantrez), as described, for example, in U.S. Pat. No. 4,627,977, to Gaffar et al., as well as, e.g., polyamino propane sulfonic acid (AMPS), diphosphonates (e.g., EHDP; AHP), polypeptides (such as polyaspartic and polyglutamic acids), and mixtures thereof.

### Tooth Substantive Agent

The present invention may include a tooth substantive agent such as polymeric surface active agents (PMSA's), which are polyelectrolytes, more specifically anionic polymers. The PMSA's contain anionic groups, e.g., phosphate, phosphonate, carboxy, or mixtures thereof, and thus, have the capability to interact with cationic or positively charged entities. The “mineral” descriptor is intended to convey that the surface activity or substantivity of the polymer is toward mineral surfaces such as calcium phosphate minerals or teeth.

PMSA's are useful in the present compositions because of their stain prevention benefit. It is believed the PMSA's provide a stain prevention benefit because of their reactivity or substantivity to mineral surfaces, resulting in desorption of portions of undesirable adsorbed pellicle proteins, in particular those associated with binding color bodies that stain teeth, calculus development and attraction of undesirable microbial species. The retention of these PMSA's on teeth can also prevent stains from accruing due to disruption of binding sites of color bodies on tooth surfaces.

The ability of PMSA's to bind stain promoting ingredients of oral care products, for example, stannous ions and cationic antimicrobials, is also believed to be helpful. The PMSA will also provide tooth surface conditioning effects which produce desirable effects on surface thermodynamic properties and surface film properties, which impart improved clean feel aesthetics both during and most importantly, following rinsing or brushing. Many of these polymeric agents are also known or expected to provide tartar control benefits when applied in oral compositions, hence providing improvement in both the appearance of teeth and their tactile impression to consumers.

The desired surface effects include: 1) creating a hydrophilic tooth surface immediately after treatment; and 2) maintaining surface conditioning effects and control of pellicle film for extended periods following product use, including post brushing or rinsing and throughout more extended periods. The effect of creating an increased hydrophilic surface can be measured in terms of a relative decrease in water contact angles. The hydrophilic surface, importantly, is maintained on the tooth surface for an extended period after using the product.

The polymeric mineral surface active agents include any agent which will have a strong affinity for the tooth surface, deposit a polymer layer or coating on the tooth surface and produce the desired surface modification effects. Suitable examples of such polymers are polyelectrolytes such as condensed phosphorylated polymers; polyphosphonates; copolymers of phosphate- or phosphonate-containing monomers or polymers with other monomers such as ethylenically unsaturated monomers and amino acids or with other polymers such as proteins, polypeptides, polysaccharides, poly(acrylate), poly(acrylamide), poly(methacrylate), poly(ethacrylate), poly(hydroxyalkylmethacrylate), poly(vinyl alcohol), poly(maleic anhydride), poly(maleate) poly(amide), poly(ethylene amine), poly(ethylene glycol), poly(propylene glycol), poly(vinyl acetate) and poly(vinyl benzyl chloride); polycarboxylates and carboxy-substituted polymers; and mixtures thereof. Suitable polymeric mineral surface active agents include the carboxy-substituted alcohol polymers described in U.S. Pat. Nos. 5,292,501; 5,213,789, 5,093,170; 5,009,882; and 4,939,284; all to Degenhardt et al. and the diphosphonate-derivatized polymers in U.S. Pat. No. 5,011,913 to Benedict et al; the synthetic anionic polymers including polyacrylates and copolymers of maleic anhydride or acid and methyl vinyl ether (e.g., Gantrez), as described, for example, in U.S. Pat. No. 4,627,977, to Gaffar et al. A preferred polymer is diphosphonate modified polyacrylic acid. Polymers with activity must have sufficient surface binding propensity to desorb pellicle proteins and remain affixed to enamel surfaces. For tooth surfaces, polymers with end or side chain phosphate or phosphonate functions are preferred although other polymers with mineral binding activity may prove effective depending upon adsorption affinity.

Additional examples of suitable phosphonate containing polymeric mineral surface active agents include the geminal diphosphonate polymers disclosed as anticalculus agents in U.S. Pat. No. 4,877,603 to Degenhardt et al; phosphonate group containing copolymers disclosed in U.S. Pat. No. 4,749,758 to Dursch et al. and in GB 1,290,724 (both assigned to Hoechst) suitable for use in detergent and cleaning compositions; and the copolymers and cotelomers disclosed as useful for applications including scale and corrosion inhibition, coatings, cements and ion-exchange resins in U.S. Pat. No. 5,980,776 to Zakikhani et al. and U.S. Pat. No. 6,071,434 to Davis et al. Additional polymers include the water-soluble copolymers of vinylphosphonic acid and acrylic acid and salts thereof disclosed in GB 1,290,724 wherein the copolymers contain from about 10% to about 90% by weight vinylphosphonic acid and from about 90% to about 10% by weight acrylic acid, more particularly wherein the copolymers have a weight ratio of vinylphosphonic acid to acrylic acid of 70% vinylphosphonic acid to 30% acrylic acid; 50% vinylphosphonic acid to 50% acrylic acid; or 30% vinylphosphonic acid to 70% acrylic acid. Other suitable polymers include the water soluble polymers disclosed by Zakikhani and Davis prepared by copolymerizing diphosphonate or polyphosphonate monomers having one or more unsaturated C═C bonds (e.g., vinylidene-1,1-diphosphonic acid and 2-(hydroxyphosphinyl)ethylidene-1,1-diphosphonic acid), with at least one further compound having unsaturated C═C bonds (e.g., acrylate and methacrylate monomers). Suitable polymers include the diphosphonate/acrylate polymers supplied by Rhodia under the designation ITC 1087 (Average MW 3000-60,000) and Polymer 1154 (Average MW 6000-55,000).

A preferred PMSA will be stable with other components of the oral care composition such as ionic fluoride and metal ions. Also preferred are polymers that have limited hydrolysis in high water content formulations, thus permitting a simple single phase dentifrice or mouthrinse formulation. If the PMSA does not have these stability properties, one option is a dual phase formulation with the polymeric mineral surface active agent separated from the fluoride or other incompatible component. Another option is to formulate non-aqueous, essentially non-aqueous or limited water compositions to minimize reaction between the PMSA and other components.

A preferred PMSA is a polyphosphate. A polyphosphate is generally understood to consist of two or more phosphate molecules arranged primarily in a linear configuration, although some cyclic derivatives may be present. Although pyrophosphates (n=2) are technically polyphosphates, the polyphosphates desired are those having around three or more phosphate groups so that surface adsorption at effective concentrations produces sufficient non-bound phosphate functions, which enhance the anionic surface charge as well as hydrophilic character of the surfaces. The inorganic polyphosphate salts desired include tripolyphosphate, tetrapolyphosphate and hexametaphosphate, among others. Polyphosphates larger than tetrapolyphosphate usually occur as amorphous glassy materials. Preferred in this invention are the linear polyphosphates having the formula:

XO(XPO₃)ₙX

wherein X is sodium, potassium or ammonium and n averages from about 3 to about 125. Preferred polyphosphates are those having n averaging from about 6 to about 21, such as those commercially known as Sodaphos (n≈6), Hexaphos (n≈13), and Glass H (n≈21) and manufactured by FMC Corporation and Astaris. These polyphosphates may be used alone or in combination. Polyphosphates are susceptible to hydrolysis in high water formulations at acid pH, particularly below pH 5. Thus it is preferred to use longer-chain polyphosphates, in particular Glass H with an average chain length of about 21. It is believed such longer-chain polyphosphates when undergoing hydrolysis produce shorter-chain polyphosphates which are still effective to deposit onto teeth and provide a stain preventive benefit.

In addition to creating surface modifying effects, the tooth substantive agent may also function to solubilize insoluble salts. For example, Glass H has been found to solubilize insoluble stannous salts. Thus, in compositions containing stannous fluoride for example, Glass H contributes to decreasing the stain promoting effect of stannous.

Other polyphosphorylated compounds may be used in addition to or instead of the polyphosphate, in particular polyphosphorylated inositol compounds such as phytic acid, myo-inositol pentakis(dihydrogen phosphate); myo-inositol tetrakis(dihydrogen phosphate), myo-inositol trikis(dihydrogen phosphate), and an alkali metal, alkaline earth metal or ammonium salt thereof. Preferred herein is phytic acid, also known as myo-inositol 1,2,3,4,5,6-hexakis (dihydrogen phosphate) or inositol hexaphosphoric acid, and its alkali metal, alkaline earth metal or ammonium salts. Herein, the term “phytate” includes phytic acid and its salts as well as the other polyphosphorylated inositol compounds.

Still other surface active organophosphate compounds useful as tooth substantive agents include phosphate mono-, di- or triesters represented by the following general structure wherein Z¹, Z², or Z³ may be identical or different, at least one being an organic moiety, preferably selected from linear or branched, alkyl or alkenyl group of from 6 to 22 carbon atoms, optionally substituted by one or more phosphate groups; alkoxylated alkyl or alkenyl, (poly)saccharide, polyol or polyether group.

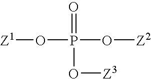

Some preferred agents include alkyl or alkenyl phosphate esters represented by the following structure:

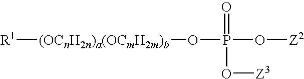

wherein R¹ represents a linear or branched, alkyl or alkenyl group of from 6 to 22 carbon atoms, optionally substituted by one or more phosphate groups; n and m, are individually and separately, 2 to 4, and a and b, individually and separately, are 0 to 20; Z² and Z³ may be identical or different, each represents hydrogen, alkali metal, ammonium, protonated alkyl amine or protonated functional alkyl amine such as an alkanolamine, or a R¹—(OCₙH₂ₙ)ₐ(OCₘH₂ₘ)_{b}— group. Examples of suitable agents include alkyl and alkyl (poly)alkoxy phosphates such as lauryl phosphate (tradenames MAP 230K and MAP 230T from Croda); PPG5 ceteareth-10 phosphate (available from Croda under the tradename Crodaphos SG); Laureth-1 phosphate (tradenames MAP L210 from Rhodia, Phosten HLP-1 from Nikkol Chemical or Sunmaep L from Sunjin); Laureth-3 phosphate (tradenames MAP L130 from Rhodia or Foamphos L-3 from Alzo or Emphiphos DF 1326 from Huntsman Chemical); Laureth-9 phosphate (tradename Foamphos L-9 from Alzo); Trilaureth-4 phosphate (tradenames Hostaphat KL 340D from Clariant or TLP-4 from Nikkol Chemical); C12-18 PEG 9 phosphate (tradename Crafol AP261 from Cognis); Sodium dilaureth-10 phosphate (tradename DLP-10 from Nikkol Chemical). Some preferred agents are polymeric, for example those containing repeating alkoxy groups as the polymeric portion, in particular 3 or more ethoxy, propoxy isopropoxy or butoxy groups.

Additional suitable polymeric organophosphate agents include dextran phosphate, polyglucoside phosphate, alkyl polyglucoside phosphate, polyglyceryl phosphate, alkyl polyglyceryl phosphate, polyether phosphates and alkoxylated polyol phosphates. Some specific examples are PEG phosphate, PPG phosphate, alkyl PPG phosphate, PEG/PPG phosphate, alkyl PEG/PPG phosphate, PEG/PPG/PEG phosphate, dipropylene glycol phosphate, PEG glyceryl phosphate, PBG (polybutylene glycol) phosphate, PEG cyclodextrin phosphate, PEG sorbitan phosphate, PEG alkyl sorbitan phosphate, and PEG methyl glucoside phosphate.

Suitable non-polymeric phosphates include alkyl mono glyceride phosphate, alkyl sorbitan phosphate, alkyl methyl glucoside phosphate, alkyl sucrose phosphates.

Compositions comprising these surface-active organophosphate compounds provide effective protection against dental erosion derived from binding of calcium minerals in teeth (hydroxyapatite) and/or deposition on the tooth surface of a protective surface coating comprised of the organophosphate compound. The protective surface coating provides control of tooth surface characteristics including modification of surface hydrophilic and hydrophobic properties and resistance to acid attack. The present surface-active organophosphate compounds may also provide desired surface conditioning effects including modifying surface hydrophilic and hydrophobic properties which can be measured in terms of changes in water contact angles, a relative decrease indicating a more hydrophilic surface and a relative increase indicating a more hydrophobic surface. It has been discovered that the surface hydrophilic and hydrophobic properties need to be balanced to optimize delivery of benefits from the present compositions comprising these surface-active organophosphate compounds. Many of the preferred organophosphate compounds also provide tartar control or antistain/whitening or surface conditioning activities, hence providing multiple clinical actions in improving overall health and structure of teeth as well as appearance and tactile impression of teeth.

The amount of tooth substantive agent will typically be from about 0.1% to about 35% by weight of the total oral composition. In dentifrice formulations, the amount is preferably from about 2% to about 30%, more preferably from about 5% to about 25%, and most preferably from about 6% to about 20%. In mouthrinse compositions, the amount of tooth substantive agent is preferably from about 0.1% to 5% and more preferably from about 0.5% to about 3%.

### Chelating Agents

Another optional agent is a chelating agent, also called sequestrants, such as gluconic acid, tartaric acid, citric acid and pharmaceutically-acceptable salts thereof. Chelating agents are able to complex calcium found in the cell walls of the bacteria. Chelating agents can also disrupt plaque by removing calcium from the calcium bridges which help hold this biomass intact. However, it is not desired to use a chelating agent which has an affinity for calcium that is too high, as this may result in tooth demineralization, which is contrary to the objects and intentions of the present invention. Suitable chelating agents will generally have a calcium binding constant of about 10¹ to 10⁵ to provide improved cleaning with reduced plaque and calculus formation. Chelating agents also have the ability to complex with metallic ions and thus aid in preventing their adverse effects on the stability or appearance of products. Chelation of ions, such as iron or copper, helps retard oxidative deterioration of finished products.

Examples of suitable chelating agents are sodium or potassium gluconate and citrate; citric acid/alkali metal citrate combination; disodium tartrate; dipotassium tartrate; sodium potassium tartrate; sodium hydrogen tartrate; potassium hydrogen tartrate; sodium, potassium or ammonium polyphosphates and mixtures thereof. The chelating agent may be used from about 0.1% to about 2.5%, preferably from about 0.5% to about 2.5% and more preferably from about 1.0% to about 2.5%.

Still other chelating agents suitable for use in the present invention are the anionic polymeric polycarboxylates. Such materials are well known in the art, being employed in the form of their free acids or partially or preferably fully neutralized water soluble alkali metal (e.g. potassium and preferably sodium) or ammonium salts. Examples are 1:4 to 4:1 copolymers of maleic anhydride or acid with another polymerizable ethylenically unsaturated monomer, preferably methyl vinyl ether (methoxyethylene) having a molecular weight (M.W.) of about 30,000 to about 1,000,000. These copolymers are available for example as Gantrez AN 139 (M.W. 500,000), AN 119 (M.W. 250,000) and S-97 Pharmaceutical Grade (M.W. 70,000), of GAF Chemicals Corporation.

Other operative polymeric polycarboxylates include the 1:1 copolymers of maleic anhydride with ethyl acrylate, hydroxyethyl methacrylate, N-vinyl-2-pyrrolidone, or ethylene, the latter being available for example as Monsanto EMA No. 1103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

Additional operative polymeric polycarboxylates are disclosed in U.S. Pat. No. 4,138,477 to Gaffar and U.S. Pat. No. 4,183,914 to Gaffar et al. and include copolymers of maleic anhydride with styrene, isobutylene or ethyl vinyl ether; polyacrylic, polyitaconic and polymaleic acids; and sulfoacrylic oligomers of M.W. as low as 1,000 available as Uniroyal ND-2.

### Surfactants

The present compositions may also comprise surfactants, also commonly referred to as sudsing agents. Suitable surfactants are those which are reasonably stable and foam throughout a wide pH range. The surfactant may be anionic, nonionic, amphoteric, zwitterionic, cationic, or mixtures thereof.

Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate (SLS) and sodium coconut monoglyceride sulfonates are examples of anionic surfactants of this type. Other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Mixtures of anionic surfactants can also be employed. Many suitable anionic surfactants are disclosed by Agricola et al., U.S. Pat. No. 3,959,458. The present composition typically comprises an anionic surfactant at a level of from about 0.025% to about 9%, from about 0.05% to about 5% or from about 0.1% to about 1%.

Another suitable surfactant is one selected from the group consisting of sarcosinate surfactants, isethionate surfactants and taurate surfactants. Preferred for use herein are alkali metal or ammonium salts of these surfactants, such as the sodium and potassium salts of the following: lauroyl sarcosinate, myristoyl sarcosinate, palmitoyl sarcosinate, stearoyl sarcosinate and oleoyl sarcosinate. The sarcosinate surfactant may be present in the present compositions from about 0.1% to about 2.5%, preferably from about 0.5% to about 2.0% by weight.

Cationic surfactants useful in the present invention include derivatives of quaternary ammonium compounds having one long alkyl chain containing from about 8 to 18 carbon atoms such as lauryl trimethylammonium chloride; cetyl pyridinium chloride; cetyl trimethylammonium bromide; coconut alkyltrimethylammonium nitrite; cetyl pyridinium fluoride; etc. Preferred compounds are the quaternary ammonium fluorides described in U.S. Pat. No. 3,535,421 to Briner et al., where said quaternary ammonium fluorides have detergent properties. Certain cationic surfactants can also act as germicides in the compositions disclosed herein. Cationic surfactants such as chlorhexidine, although suitable for use in the current invention, are not preferred due to their capacity to stain the oral cavity's hard tissues. Persons skilled in the art are aware of this possibility and should incorporate cationic surfactants with this limitation in mind.

Nonionic surfactants that can be used in the compositions of the present invention include compounds produced by the condensation of alkylene oxide groups (hydrophilic in nature) with an organic hydrophobic compound which may be aliphatic or alkylaromatic in nature. Examples of suitable nonionic surfactants include the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials.

Zwitterionic synthetic surfactants useful in the present invention include derivatives of aliphatic quaternary ammonium, phosphonium, and sulfonium compounds, in which the aliphatic radicals can be straight chain or branched, and wherein one of the aliphatic substituents contains from about 8 to 18 carbon atoms and one contains an anionic water-solubilizing group, e.g., carboxy, sulfonate, sulfate, phosphate or phosphonate.

Suitable betaine surfactants are disclosed in U.S. Pat. No. 5,180,577 to Polefka et al. Typical alkyl dimethyl betaines include decyl betaine or 2-(N-decyl-N,N-dimethylammonio) acetate, coco betaine or 2-(N-coco-N,N-dimethyl ammonio) acetate, myristyl betaine, palmityl betaine, lauryl betaine, cetyl betaine, cetyl betaine, stearyl betaine, etc. The amidobetaines are exemplified by cocoamidoethyl betaine, cocoamidopropyl betaine, and lauramidopropyl betaine.

### Miscellaneous Carrier Materials

Water employed in the preparation of commercially suitable oral compositions should preferably be of low ion content and free of organic impurities. Water may comprise up to about 99% by weight of the aqueous compositions herein. These amounts of water include the free water which is added plus that which is introduced with other materials, such as with sorbitol.

The present invention may also include an alkali metal bicarbonate salt, which may serve a number of functions including abrasive, deodorant, buffering and adjusting pH. Alkali metal bicarbonate salts are soluble in water and unless stabilized, tend to release carbon dioxide in an aqueous system. Sodium bicarbonate, also known as baking soda, is a commonly used alkali metal bicarbonate salt. The present composition may contain from about 0.5% to about 30% by weight of an alkali metal bicarbonate salt.

The pH of the present compositions may be adjusted through the use of buffering agents. Buffering agents, as used herein, refer to agents that can be used to adjust the pH of aqueous compositions such as mouthrinses and dental solutions preferably to a range of about pH 4.0 to about pH 8.0. Buffering agents include sodium bicarbonate, monosodium phosphate, trisodium phosphate, sodium hydroxide, sodium carbonate, sodium acid pyrophosphate, citric acid, and sodium citrate and are typically included at a level of from about 0.5% to about 10% by weight.

Poloxamers may be employed in the present compositions. A poloxamer is classified as a nonionic surfactant and may also function as an emulsifying agent, binder, stabilizer, and other related functions. Poloxamers are difunctional block-polymers terminating in primary hydroxyl groups with molecular weights ranging from 1,000 to above 15,000. Poloxamers are sold under the tradename of Pluronics and Pluraflo by BASF including Poloxamer 407 and Pluraflo L4370.

Other emulsifying agents that may be used include polymeric emulsifiers such as the Pemulen® series available from B.F. Goodrich, and which are predominantly high molecular weight polyacrylic acid polymers useful as emulsifiers for hydrophobic substances.

Titanium dioxide may also be added to the present compositions as coloring or opacifying agent typically at a level of from about 0.25% to about 5% by weight.

Other optional agents that may be used in the present compositions include dimethicone copolyols selected from alkyl- and alkoxy-dimethicone copolyols, such as C12 to C20 alkyl dimethicone copolyols and mixtures thereof, as aid in providing positive tooth feel benefits. Highly preferred is cetyl dimethicone copolyol marketed under the trade name Abil EM90. The dimethicone copolyol is generally present from about 0.01% to about 25%, preferably from about 0.1% to about 5%, more preferably from about 0.5% to about 1.5% by weight.

### Method of Use

The present invention also relates to methods for cleaning, refreshing and controlling bacterial activity in the oral cavity which cause undesirable conditions including plaque, caries, calculus, gingivitis, periodontal disease and malodor. The benefits of these compositions may increase over time when the composition is used repeatedly.

The method of use or treatment herein may comprise contacting a subject's dental enamel surfaces and mucosa in the mouth with the oral compositions according to the present invention. The method may comprise brushing with a dentifrice or rinsing with a dentifrice slurry or mouthrinse. Other methods include contacting the topical oral gel, denture product, mouthspray, or other form with the subject's teeth and oral mucosa. The subject may be any person or animal whose tooth surface and oral cavity are contacted with the oral composition. By animal is meant to include household pets or other domestic animals, or animals kept in captivity.

For example, a method of treatment may include a person brushing a dog's teeth with one of the dentifrice compositions. Another example would include rinsing a cat's mouth with an oral composition for a sufficient amount of time to see a benefit.

### EXAMPLES

The following examples further describe and demonstrate embodiments within the scope of the present invention. These examples are given solely for the purpose of illustration and are not to be construed as limitations of the present invention as many variations thereof are possible without departing from the spirit and scope.

### Dentifrice Compositions

Dentifrice compositions according to the present invention 1A-1F and 2A-2F are shown below with amounts of ingredients in weight %. These compositions are made using conventional methods. In consumer sensory tests, these compositions are rated as having characteristics typical of normal dentifrice products including cleaning ability, mouthfeel, stand-up, cleanliness, etc. The dispersibility characteristics of Examples 2A-2F were compared with currently marketed products using the method described previously. Results, i.e., time to disperse a sample of product, are shown below. All dentifrice compositions according to the present invention containing carrageenan thickening agent with little or no humectant dispersed more quickly than comparative commercial products which contained much higher levels of humectant.

| Component | Ex. 1A | Ex. 1B | Ex. 1C | Ex. 1D | Ex. 1E | Ex. 1F |
|---|---|---|---|---|---|---|
| USP Water | 74.129 | 64.129 | 67.207 | 67.917 | 60.627 | 46.17 |
| Sorbitol Soln (70%) | | 10.00 | | | | |
| Glycerin | | | 7.00 | | | |
| Saccharin Sodium, USP | 0.30 | 0.30 | 0.30 | 0.30 | 0.48 | 0.48 |
| Sodium Fluoride; USP | 0.321 | 0.321 | 0.243 | 0.243 | | |
| Sodium MFP | | | | | 0.80 | 0.80 |
| Sodium Acid Pyrophosphate FCC | | | | 1.26 | | |
| Titanium Dioxide USP | 0.25 | 0.25 | 0.25 | 0.25 | 0.25 | 0.25 |
| Silica Abrasive, NF (Zeodent 119) | 15.00 | 15.00 | 15.00 | 15.00 | | |
| Calcium Carbonate, USP | | | | | 25.00 | 42.00 |
| Thickening Silica NF (Zeodent 165) | 3.00 | 3.00 | 2.00 | 4.00 | 3.00 | 0.50 |
| Na Carboxymethylcellulose | 0.50 | 0.50 | | 0.50 | 0.40 | 0.40 |
| Carrageenan¹ | 1.50 | 1.50 | 2.00 | 1.50 | 2.00 | 1.40 |
| Flavor | 1.00 | 1.00 | 1.00 | 1.00 | 1.00 | 1.00 |
| Sodium Hydroxide (50% Soln) | | | | 0.70 | | |
| Sodium Lauryl Sulfate 28% Soln | 4.00 | 4.00 | 5.00 | 4.00 | 7.00 | 7.00 |
| Sodium Laureth-1 Phosphate 30% Soln | | | | 3.33 | | |

¹Supplied by FMC under the Viscarin ® tradename.

| Component | Ex. 2A | Ex. 2B | Ex. 2C | Ex. 2D | Ex. 2E | Ex. 2F |
|---|---|---|---|---|---|---|
| USP Water | 73.457 | 62.427 | 69.297 | 64.712 | 65.742 | 67.376 |
| Sorbitol Soln (70%) | | 7.648 | 1.752 | 4.878 | 6.174 | 3.652 |
| Saccharin Sodium, USP | 0.30 | 0.30 | 0.30 | 0.30 | 0.30 | 0.30 |
| Sodium Fluoride; USP | 0.243 | 0.243 | 0.243 | 0.243 | 0.243 | 0.243 |
| Disodium Pyrophosphate | 0.25 | 1.26 | 1.26 | 1.26 | 1.26 | 1.26 |
| Titanium Dioxide USP | 0.25 | 0.25 | 0.25 | 0.25 | 0.25 | 0.25 |
| Silica Abrasive, NF (Zeodent 119) | 15.00 | 17.375 | 15.061 | 19.735 | 15.399 | 16.711 |
| Sodium Hydroxide (50% Soln) | 0.50 | 0.70 | 0.70 | 0.700 | 0.70 | 0.70 |
| Thickening Silica NF (Zeodent 165) | 3.00 | 2.892 | 5.598 | 2.255 | 4.446 | 3.98 |
| Na Carboxymethylcellulose | 0.50 | 0.137 | 0.002 | 0.357 | 0.014 | 0.012 |
| Carrageenan¹ | 1.50 | 1.768 | 1.747 | 1.52 | 1.682 | 1.726 |
| Flavor | 1.00 | 1.00 | 1.00 | 1.00 | 1.00 | 1.00 |
| Sodium Lauryl Sulfate 28% Soln | 4.00 | 4.00 | 4.00 | 4.00 | 4.00 | 4.00 |

¹Supplied by FMC under the Viscarin ® tradename.

| | | Dispersibility |
|---|---|---|
| Product | Lot Code | (min.) |
| Ex 2A | | 17.0 |
| Ex 2B | | 16.5 |
| Ex 2C | | 14.75 |
| Ex 2D | | 20.75 |
| Ex 2E | | 19.25 |
| Ex 2F | | 17.25 |
| Darlie Green Tea Gel | 20080508 60354 | 20.0 |
| Crest ® Pro Health Night | 818336 MA10 | 21.0 |
| Colgate ® MaxFresh | ABR07 5094 MX1125 | 24.5 |
| Cristales Frefrescantes | | |
| Cinnamint | | |
| Colgate ® Triple Accion | 7019 MX1134 | 25.0 |
| Maxima Proteccion Anticaries | | |
| Colgate ® Total Advanced | 21FEB092 | 40.0 |
| Clean Plus Whitening Gel | | |
| Aquafresh ® Extreme Clean | 04/2010 8D21 | 50.0 |
| Polishing Action | | |
| Aquafresh ® Mild & Minty | 485G | 50.0 |
| Zhong Hua 5 | 20090527C1 | 50.0 |
| Crest ® Calci-Dent Proteccion | 70652709A2 | 50.0 |
| Familiar | | |
| Colgate ® Chinese Label Stripe | 20071105 1201 | 50.0 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oral care composition having improved dispersibility during use comprising
(a) a thickening/binding agent comprising from about 0.1% to about 10% by weight of the oral care composition of carrageenan that provides a water viscosity of at least about 20 mPa·s in a 1.5% solution at 25° C. and effective water-binding capacity to prevent significant water loss from the composition when exposed to air to cause unacceptable drying out, and
(b) an orally acceptable carrier comprising no more than about 10% by weight of humectant(s) and one or more other oral care agents.

2. An oral care composition in the form of a dentifrice having improved dispersibility during use comprising
(a) a thickening/binding agent comprising from about 0.1% to about 10% by weight of carrageenan that provides a water viscosity of at least about 20 mPa·s in a 1.5% solution at 25° C. and effective water-binding capacity such that there is no more than about 0.75% water loss from the dentifrice composition when exposed to air for 30 minutes at room temperature conditions and 50% relative humidity, and
(b) an orally acceptable carrier comprising no more than about 10% by weight of humectant(s) and one or more other oral care agents.

3. An oral care composition according to claim 1, wherein the orally acceptable carrier comprises one or more of oral care agents selected from abrasive, anticaries agent, fluoride ion source, antimicrobial/antiplaque agent, bleaching agent, anticalculus agent, desensitizing agent, tooth substantive agent, chelating agent, surfactant or flavor system.

4. An oral care composition according to claim 1 further comprising an additional thickening/binding agent selected from one or a combination of carboxyvinyl polymers, natural and synthetic clays, hydroxyethyl cellulose (HEC), carboxymethylcellulose (CMC), gum karaya, xanthan gum, gum arabic, gum tragacanth, magnesium aluminum silicate or finely divided silica.

5. An oral care composition according to claim 1 wherein the humectant when present comprises one or a mixture of glycerin, sorbitol, xylitol, butylene glycol, polyethylene glycol, propylene glycol and trimethyl glycine

6. An oral care composition according to claim 3 wherein the abrasive comprises one or a mixture of silica, calcium carbonate, sodium polymetaphosphate, dicalcium phosphate dihydrate or calcium pyrophosphate.

7. An oral care composition according to claim 3 wherein the fluoride ion source comprises one or a mixture of sodium fluoride, stannous fluoride, indium fluoride, amine fluoride or sodium monofluorophosphate.

8. An oral care composition according to claim 3 wherein the tooth substantive agent comprises one or a mixture of phosphate compounds selected from polyphosphates having an average chain length of 3 or more, phytates, alkyl phosphates or alkyl (poly)alkoxy phosphates.

9. An oral care composition according to claim 3 wherein the anticalculus agent comprises a pyrophosphate salt.

10. A method for improving dispersibility of dentifrice compositions comprising formulating the dentifrice with a thickening/binding agent comprising from about 0.1% to about 10% by weight of carrageenan that provides a water viscosity of at least about 20 mPa·s in a 1.5% solution at 25° C. and effective water-binding capacity such that there is no more than about 0.75% water loss from the dentifrice composition when exposed to air for 30 minutes at room temperature conditions and 50% relative humidity and an orally acceptable carrier comprising no more than about 10% by weight of humectant(s) and one or more other oral care agents, wherein the dentifrice essentially disperses completely during a typical brushing period.
